(19) United States
(12) Patent Application Publication
(10) Pub. No.: US 2011/0272685 A1
(43) Pub. Date: Nov. 10, 2011

(54) MATERIALS FOR ORGANIC ELECTROLUMINESCENCE DEVICES

(75) Inventors: Amir Hossain Parham, Frankfurt (DE); Christof Pflumm, Frankfurt (DE); Philipp Stoessel, Frankfurt (DE); Holger Heil, Frankfurt (DE); Arne Buesing, Frankfurt (DE)

(73) Assignee: Merck Patent GmbH, Darmstadt (DE)

(21) Appl. No.: 13/145,023

(22) PCT Filed: Dec. 22, 2009
(86) PCT No.: PCT/EP09/09221

§ 371 Date: Jul. 18, 2011

(30) Foreign Application Priority Data
Jan. 20, 2009 (DE) 10 2009 005 288.7

(51) Int. Cl. H01L 51/54; C07D 213/89; C07C 321/26; C07D 333/52; C07C 13/573; C07C 211/54; C07D 487/14

(52) U.S. Cl. 257/40; 564/305; 546/6; 544/247; 549/43; 585/26; 568/58; 257/E51.024

(57) ABSTRACT

The present invention describes indenofluorene derivatives containing a heteroaromatic bridge atom as a novel class of materials having emitting and hole-transporting properties, in particular for use in the emission and/or charge-transport layer of electroluminescent devices. The invention furthermore relates to a process for the preparation of the compounds according to the invention and to electronic devices comprising same.

The present invention describes indenofluorene derivatives containing a heteroaromatic bridge atom as a novel class of materials having emitting and hole-transporting properties, in particular for use in the emission and/or charge-transport layer of electroluminescent devices. The invention furthermore relates to a process for the preparation of the compounds according to the invention and to electronic devices comprising same.

The general structure of organic electroluminescent devices is described, for example, in U.S. Pat. No. 4,539,507, U.S. Pat. No. 5,151,629, EP 0676461 and WO 98/27136. However, these devices still show a need for improvement:
1. The efficiency is still low, especially in the case of fluorescent OLEDs, and should be improved.
2. The operating lifetime is frequently still short, especially in the case of blue emission, and consequently there is a further need for improvement here.
3. The operating voltage is fairly high, both in the case of fluorescent and in the case of phosphorescent OLEDs. A reduction in the operating voltage results in an improvement in the power efficiency. This is of major importance, in particular, for mobile applications.
4. In the case of hole-transport materials in accordance with the prior art, the voltage is dependent on the layer thickness of the hole-transport layer. In practice, a thicker layer thickness of the hole-transport layer would frequently be desirable in order to improve the optical coupling-out and the production yield. However, this cannot be achieved with materials in accordance with the prior art owing to the accompanying increase in voltage. There therefore continues to be a need for improvement here.
5. Some materials, in particular hole-transport materials, in accordance with the prior art have the problem that they crystallise at the edge of the vapour-deposition source during the vapour-deposition process and thus clog the vapour-deposition source. Materials which can be processed better are therefore desirable for mass production.

Indenofluorenamines are used as charge-transport materials and -injection materials owing to very good hole mobility. This class of materials exhibits a comparatively low dependence of the voltage on the thickness of the transport layer. EP 1860097, WO 2006/100896, DE 102006025846, WO 006/122630 and WO 2008/006449 disclose indenofluorenediamines for use in electronic devices. Good lifetimes on use as hole-transport material or as dark-blue emitters are cited therein. However, these compounds have the problem that, due to the crystallinity of the materials, they exhibit problematic behaviour during vapour deposition in mass production since the materials crystallise on the vapour-deposition source during vapour deposition and clog it. The use of these materials in production is therefore associated with increased technical complexity. Further improvements are therefore still desirable here.

There continues to be a demand, in particular, for improved emitting compounds, in particular blue-emitting compounds, which result in good efficiencies and at the same time in long lifetimes in organic electroluminescent devices and which can be processed without problems in industry. This applies equally to charge-transport and -injection compounds and to matrix materials for fluorescent or phosphorescent compounds. In particular, there is a need for improvement in the crystallinity of the materials.

The object of the present invention thus consists in the provision of such compounds.

Surprisingly, it has been found that electroluminescent devices which use indenofluorene derivatives containing precisely one heteroaromatic bridge atom have significant improvements over the prior art, in particular on use as blue-emitting dopants in a host material or as hole-transport compounds. On use as hole-transport compounds, replacement of a carbon atom by a heteroatom in one of the two bridges can enable a reduction in crystallinity and thus improved processability to be achieved. Furthermore, lower operating voltages owing to changes in the interfacial morphology and a lower dependence of the voltage on the transport layer thickness, possibly owing to improved hole mobility, arise. On use as dark-blue dopants, the introduction of heteroaromatic bridge atoms results in a longer lifetime and improved efficiency.

To this end, the invention provides a compound of the general formula I or II

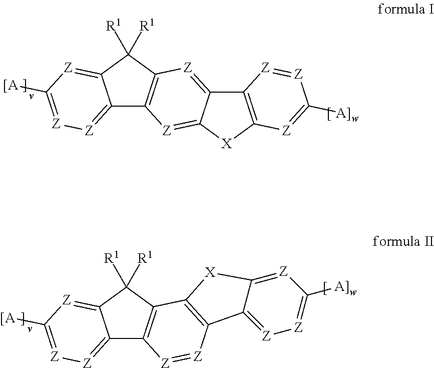

where
A corresponds to the general formula III

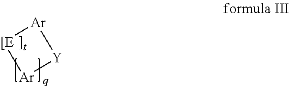

and where the link to the compound of the general formula I or II takes place via Y;
Y is in each case, independently of one another, N, P, P═O, B, C═O, O, S, S═O or SO₂;
Z is in each case, independently of one another, CR or N;
X is in each case, independently of one another, a divalent bridge selected from B(R¹), C═O, C═C(R¹)₂, S, S═O, SO₂ and N(R¹);
R is in each case, independently of one another, H, D, F, Cl, Br, I, N(Ar)₂, N(R²)₂, C(═O)Ar, P(═O)Ar₂, S(═O)Ar, S(═O)₂Ar, CR²═CR²Ar, CN, NO₂, Si(R²)₃, B(OR²)₂, OSO₂R², a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which may be substituted by one or more radicals R², where one or more non-adjacent CH₂ groups may be replaced by R²C═CR², C≡C, Si(R²)₂, Ge(R²)₂, Sn(R²)₂, C═O, C═S, C═Se, C═NR², P(═O)(R²), SO, SO₂, NR², O, S or CONR² and where one or more H atoms may be replaced by D, F, Cl, Br, I, CN or NO₂, or an aromatic or heteroaromatic ring system having 5 to 40 aromatic ring atoms, which may in each case be substituted by one or more radicals R², or an aryloxy or heteroaryloxy group having 5 to 40 aromatic ring atoms, which may be substituted by one or more radicals R², or a combination of these systems; where, in addition, two or more substituents R may also form a mono- or polycyclic aliphatic ring system with one another;
R¹ is in each case, independently of one another, H, D, F, Cl, Br, I, CN, NO₂, N(R²)₂, B(OR²)₂, Si(R²)₃, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which may be substituted by one or more radicals R², where one or more non-adjacent CH₂ groups may be replaced by —R²C═CR²—, —C≡C—, Si(R²)₂, Ge(R²)₂, Sn(R²)₂, C═O, C═S, C═Se, C═NR², —O—, —S—, —COO— or —CONR²— and where one or more H atoms may be replaced by D, F, Cl, Br, I, CN or NO₂, or arylamines or substituted carbazoles, each of which may be substituted by one or more radicals R², or an aromatic or heteroaromatic ring system having 5 to 40 aromatic ring atoms, which may be substituted by one or more non-aromatic radicals R², or an aryloxy or heteroaryloxy group having 5 to 40 aromatic ring atoms, which may be substituted by one or more non-aromatic radicals R², or a combination of these systems, where, in addition, two or more substituents R¹ may also form a mono- or polycyclic ring system with one another;
R² is in each case, independently of one another, H, D or an aliphatic or aromatic hydrocarbon radical having 1 to 20 C atoms;
Ar is in each case, independently of one another, an aromatic or heteroaromatic ring system having 5 to 40 aromatic ring atoms, which may be substituted by one or more radicals R¹;
E is in each case, independently of one another, a single bond, N(R¹), O, S, C(R¹)₂, Si(R¹)₂ or B(R¹);
q=1 if the corresponding central atom of the group Y is an element from main group 3 or 5 or =0 if the corresponding central atom of the group Y is an element from main group 4 or 6;
t is in each case, independently of one another, 0 or 1, with the proviso that t=0 if q=0, and where t=0 means that a radical R¹ is bonded instead of the group E;
v is in each case, independently of one another, 0 or 1, with the proviso that the sum of v and w is greater than or equal to one, where v=0 means that a radical R is bonded instead of A;
w is in each case, independently of one another, 0 or 1, with the proviso that the sum of v and w is greater than or equal to one, where w=0 means that a radical R is bonded instead of A.

In an embodiment of the invention, it is preferred for the radical Y in the compounds of the general formula I or II in each case to be N or C═O.

It is furthermore preferred for X to be selected from N(R¹) or S, where R¹ has the meaning indicated above.

In still a further embodiment of the invention, it is preferred for the group Z to be in each case, independently of one another, CR. The radical R here preferably has the meaning indicated above.

In still a further embodiment of the invention, it is preferred for Ar in the compounds of the general formula I or II to be phenyl, naphthyl, a substituted aromatic or heteroaromatic ring system having 5-15 carbon atoms or an aromatic or heteroaromatic ring system which is substituted by arylamine or carbazole.

In still a further embodiment of the invention, E is not present, i.e. t=0, or E is a single bond or C(R¹)₂, where R¹ has the meaning indicated above.

It is furthermore preferred for the following to apply in the compounds of the general formula I or II: v=w=1 or v=0 and w=1 or v=1 and w=0.

In a further embodiment of the invention, the compound is selected from the formula Ia or IIa:

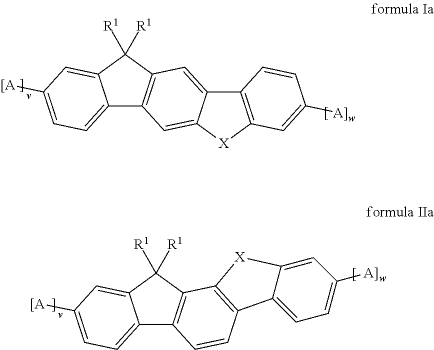

where the symbols and indices have the meanings indicated above. X is particularly preferably equal to S or N(R¹).

It is furthermore preferred for the compounds of the general formula I or II to satisfy the following structural formulae 1 to 72:

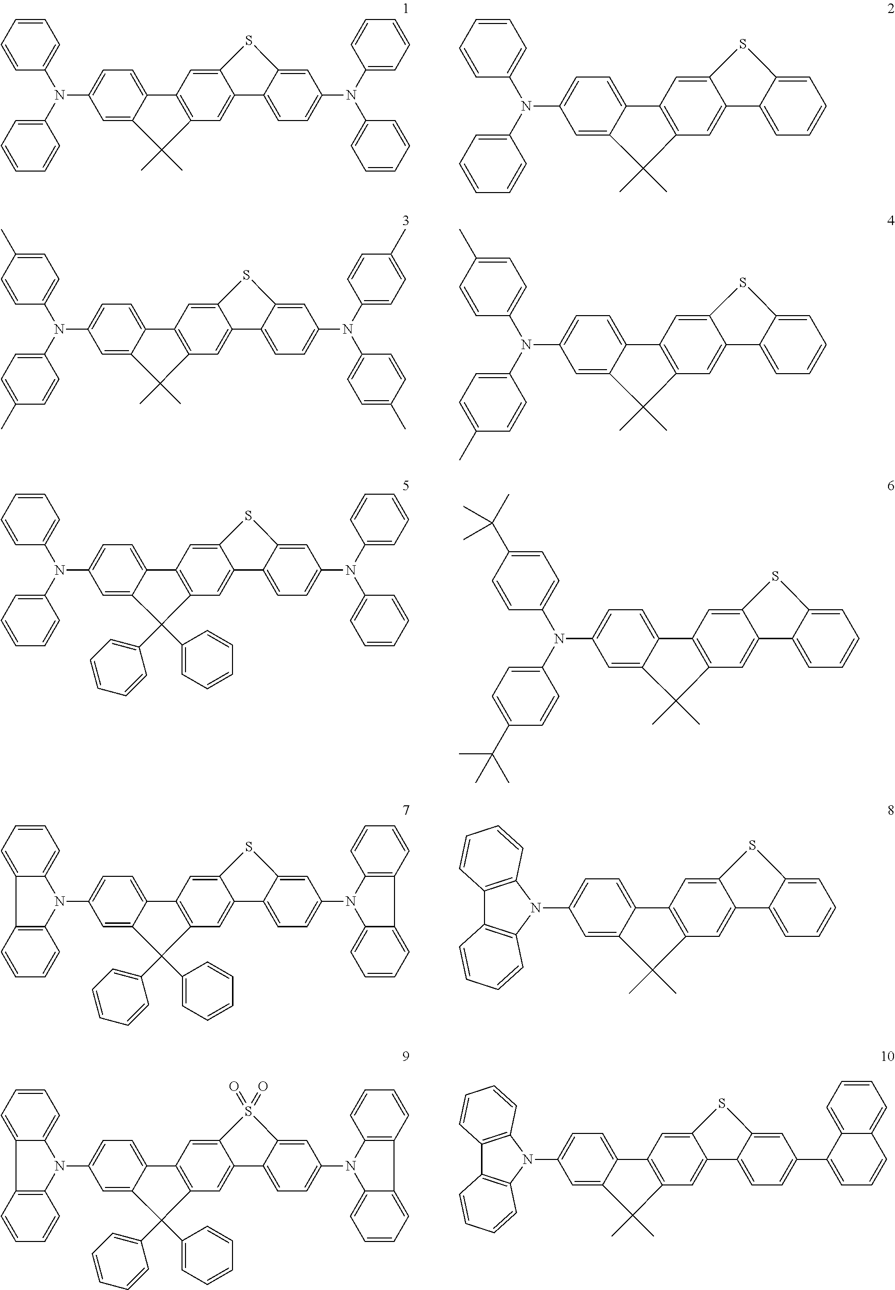

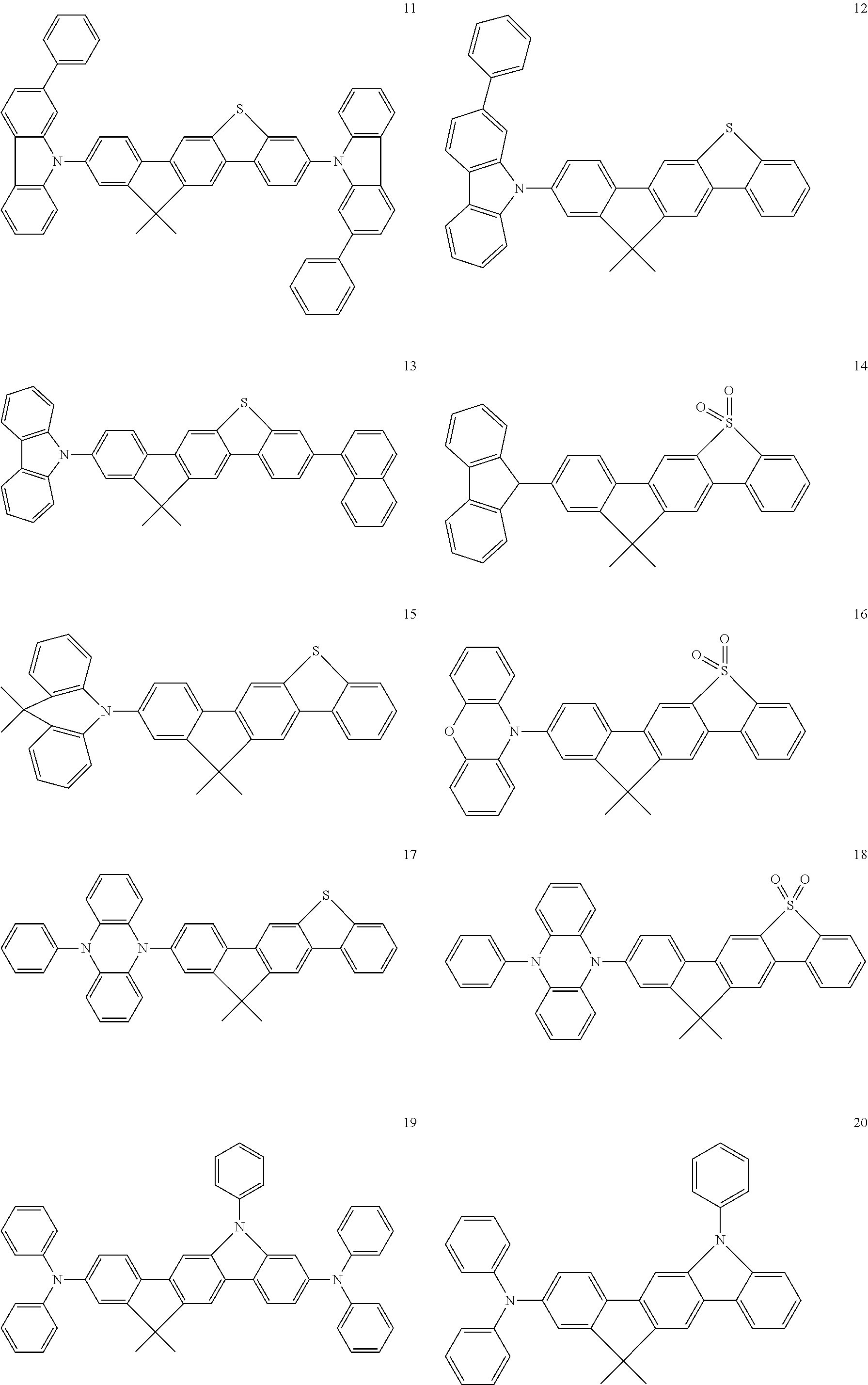

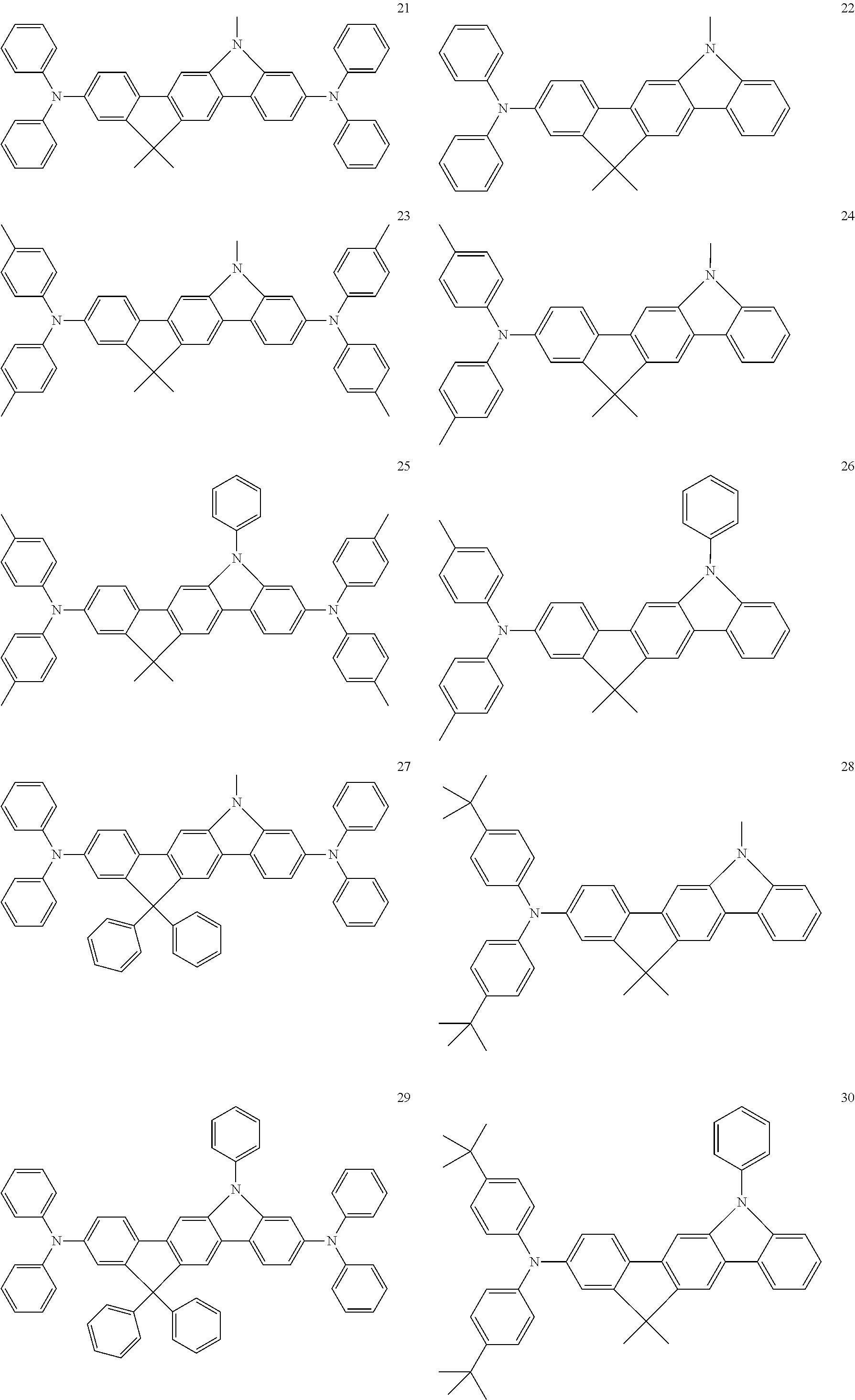

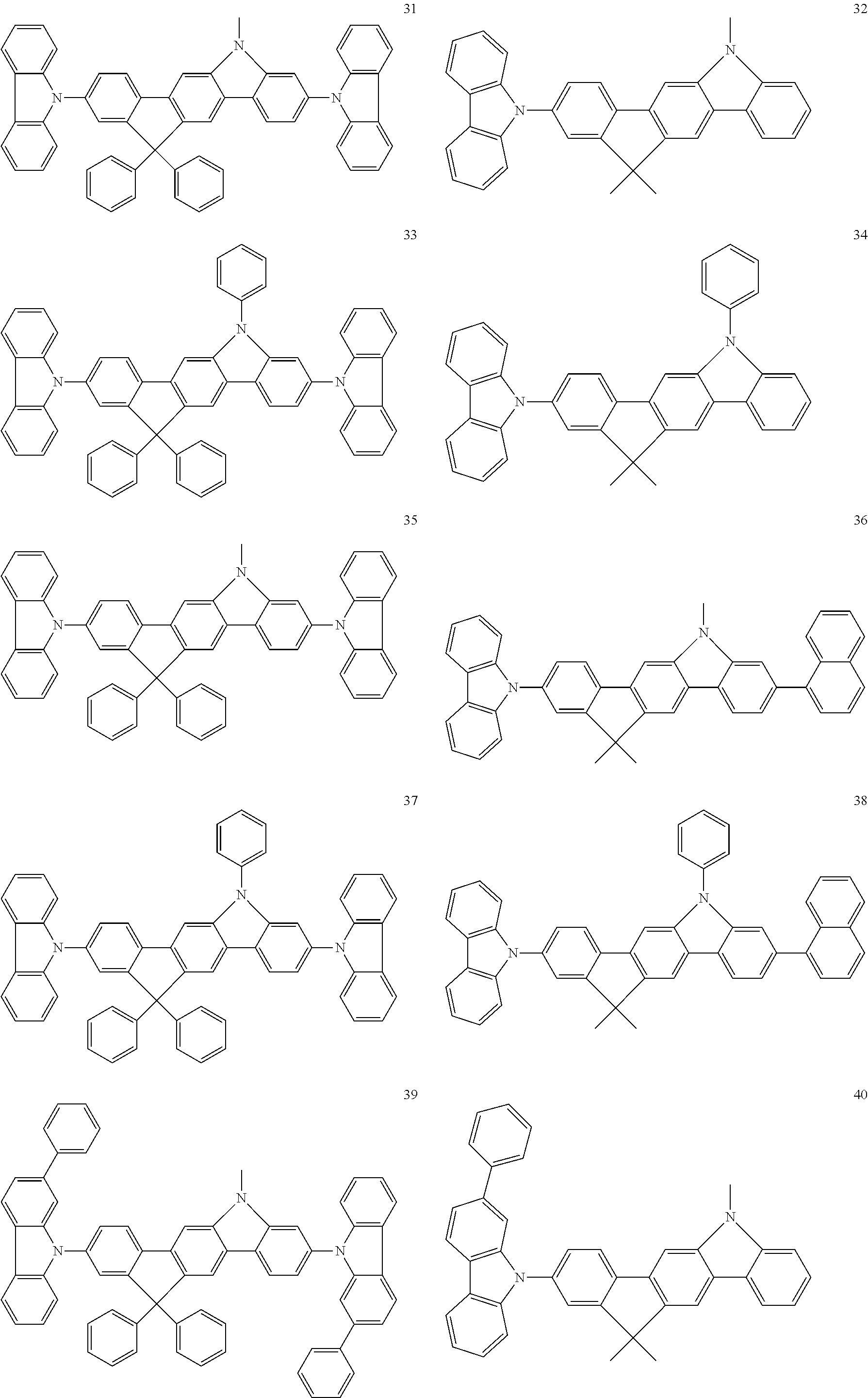

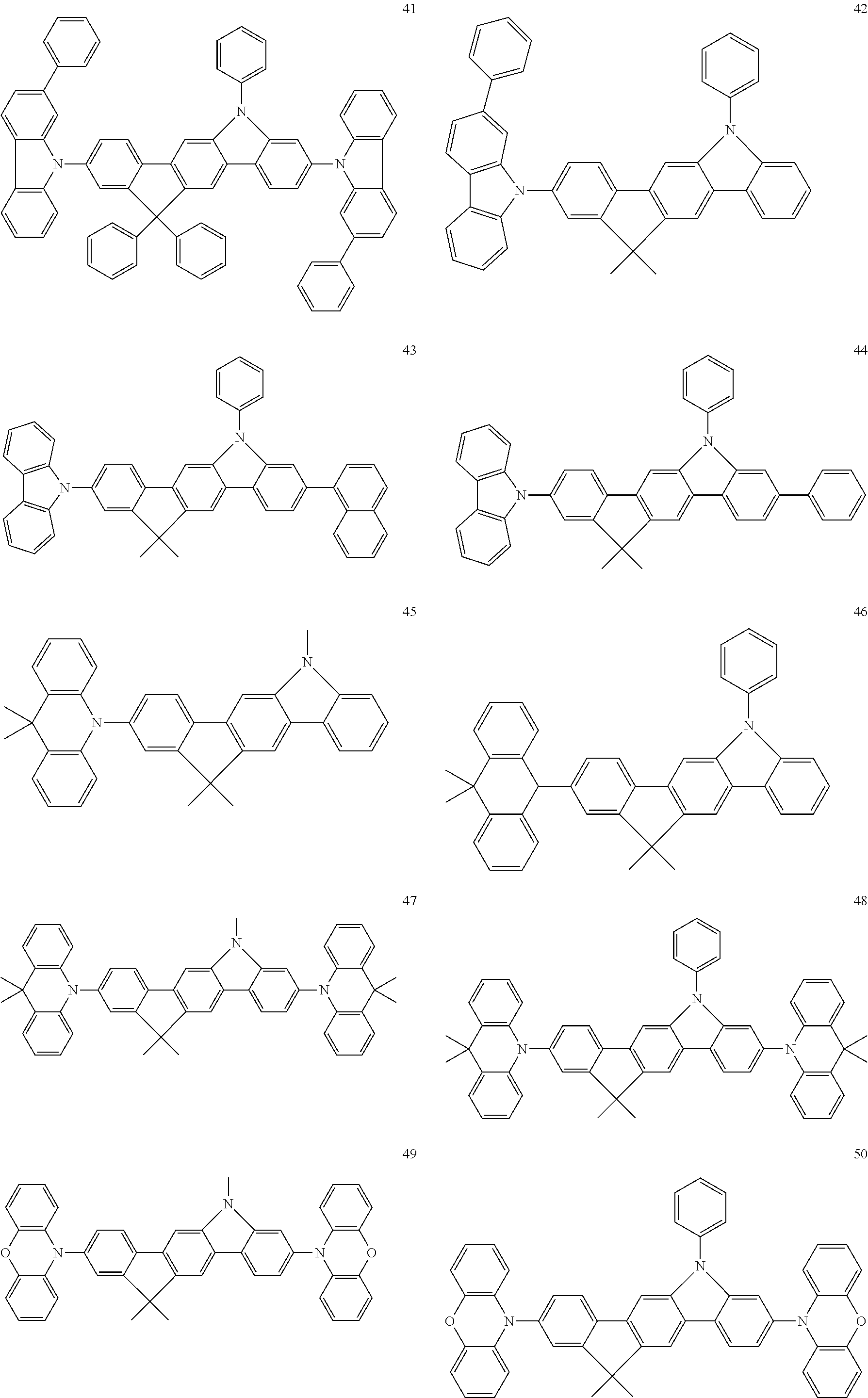

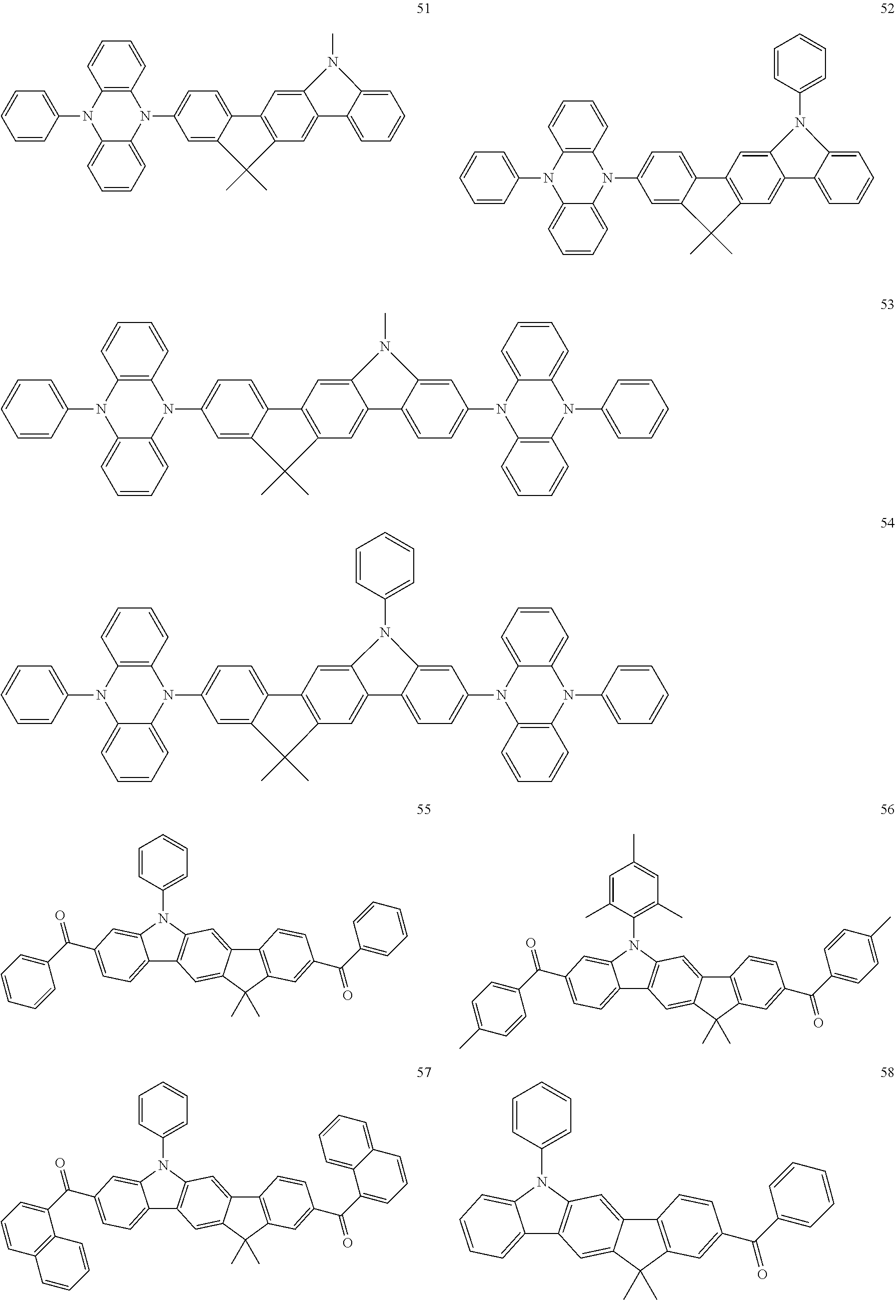

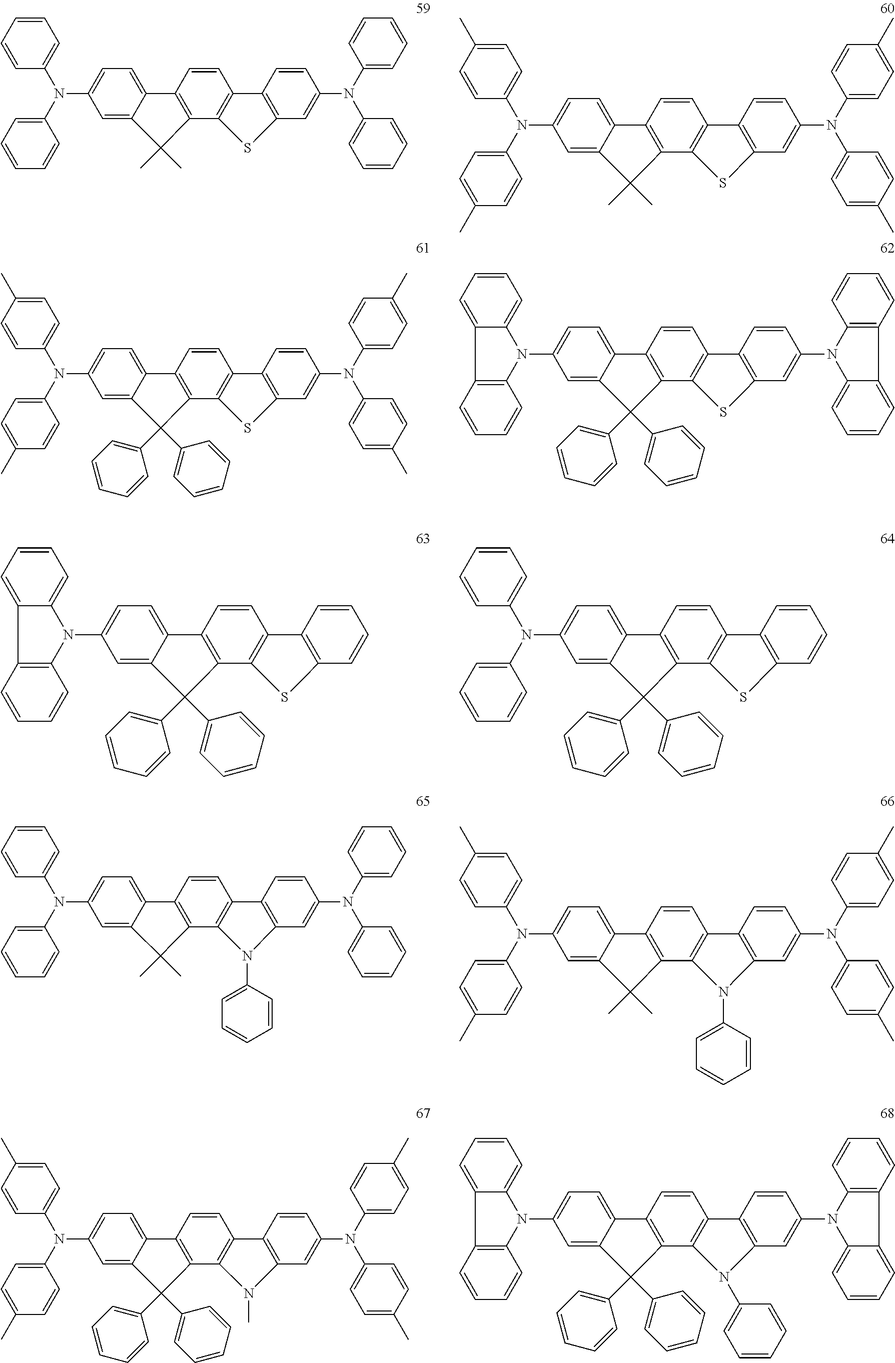

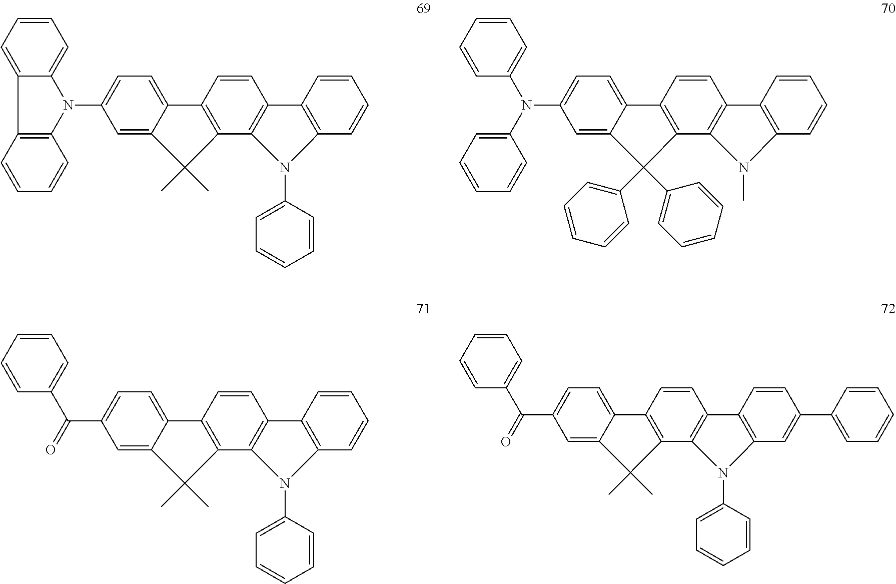

For the purposes of the present invention, an alkyl group having 1 to 40 C atoms, in which, in addition, individual H atoms or CH₂ groups may be substituted by the groups mentioned above, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl and 2,2,2-trifluoroethyl. For the purposes of this invention, an alkenyl group is taken to mean, in particular, ethenyl, propenyl, butenyl, pentenyl, cyclopentenyl, hexenyl, cyclohexenyl, heptenyl, cycloheptenyl, octenyl or cyclooctenyl. For the purposes of this invention, an alkynyl group is taken to mean, in particular, ethynyl, propynyl, butynyl, pentynyl, hexynyl or octynyl. A C₁- to C₄₀-alkoxy group is preferably taken to mean methoxy, trifluoromethoxy, ethoxy, n-propoxy, i-propoxy, n-butoxy, i-butoxy, s-butoxy, t-butoxy or 2-methylbutoxy.

For the purposes of this invention, an aryl group preferably contains 5 to 40 C atoms; for the purposes of this invention, a heteroaryl group contains 2 to 40 C atoms and at least one heteroatom, with the proviso that the sum of C atoms and heteroatoms is at least 5. The heteroatoms are preferably selected from N, O and/or S. An aryl group or heteroaryl group here is taken to mean either a simple aromatic ring, i.e. benzene, or a simple heteroaromatic ring, for example pyridine, pyrimidine, thiophene, etc., or a condensed aryl or heteroaryl group, for example naphthalene, anthracene, phenanthrene, quinoline, isoquinoline, benzothiophene, benzofuran and indole, etc.

For the purposes of this invention, an aromatic ring system contains 5 to 40 C atoms in the ring system. For the purposes of this invention, a heteroaromatic ring system contains 2 to 40 C atoms and at least one heteroatom in the ring system, with the proviso that the sum of C atoms and heteroatoms is at least 5. The heteroatoms are preferably selected from N, O and/or S. For the purposes of this invention, an aromatic or heteroaromatic ring system is intended to be taken to mean a system which does not necessarily contain only aryl or heteroaryl groups, but instead in which a plurality of aryl or heteroaryl groups may also be interrupted by a non-aromatic unit (preferably less than 10% of the atoms other than H), such as, for example, an sp³-hybridised C, N or O atom. Thus, for example, systems such as 9,9′-spirobifluorene, 9,9-diarylfluorene, triarylamine, diaryl ether, stilbene, etc. are also intended to be taken to be aromatic ring systems for the purposes of this invention, as are systems in which two or more aryl groups are interrupted, for example, by a linear or cyclic alkyl group or by a silyl group.

An aromatic or heteroaromatic ring system having 5-60 aromatic ring atoms, which may also in each case be substituted by the above-mentioned radicals R and which may be linked to the aromatic or heteroaromatic ring system via any desired positions, is taken to mean, in particular, groups derived from benzene, naphthalene, anthracene, phenanthrene, pyrene, chrysene, benzanthracene, perylene, fluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The compounds according to the invention can be prepared by synthetic steps known to the person skilled in the art, such as, for example, bromination, Suzuki coupling, Hartwig-Buchwald coupling, etc. The synthesis of derivatives containing nitrogen as bridge atom X is shown in general terms in Scheme 1.

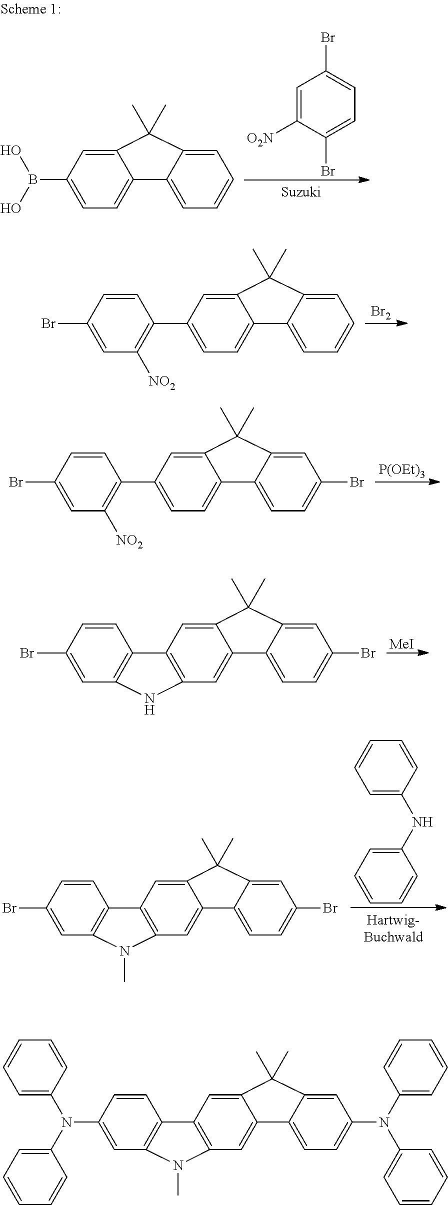

The synthesis proceeds from a fluorene-2-boronic acid derivative, which is coupled to 1,4-dibromo-2-nitrobenzene in a Suzuki coupling. This may be followed by a halogenation, for example a bromination, at the fluorene unit. The nitro group is cyclised under the action of a phosphite, for example triethyl phosphite, giving the corresponding indenocarbazole derivative. The nitrogen can then be alkylated by means of alkylating agents or arylated in a Hartwig-Buchwald reaction. In a final step, the reactive leaving groups, for example the bromine groups, are reacted to give the desired molecule. This can be carried out, for example, in a Hartwig-Buchwald coupling to give the corresponding amine. Ketones, phosphine oxides, etc., are obtainable by metallation, for example lithiation, and reaction with an electrophile. The structures here may of course also be substituted by further substituents.

The synthesis of derivatives containing sulfur as bridge atom X is shown in general terms in Scheme 2.

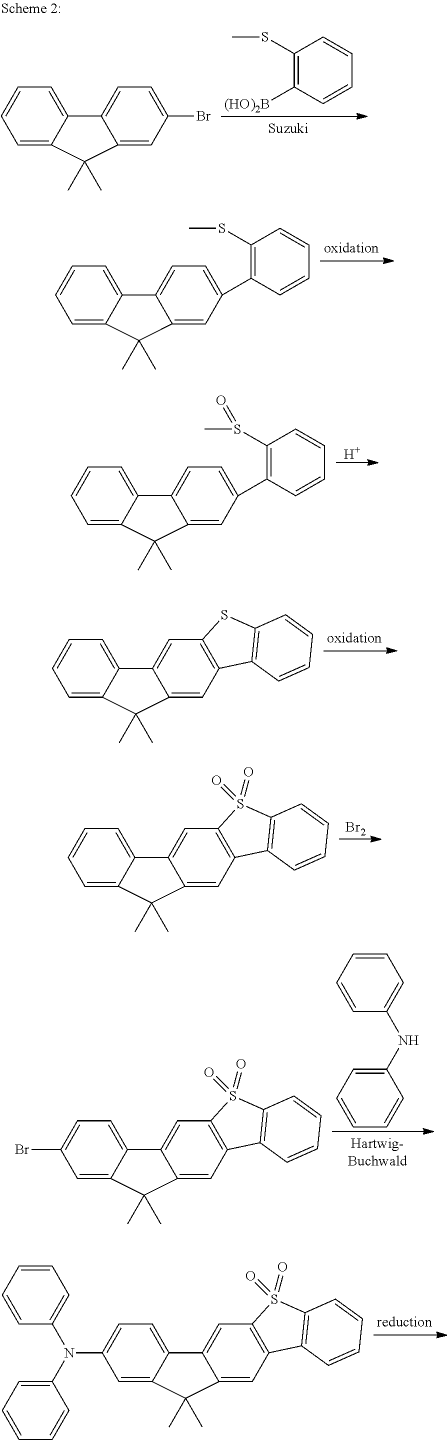

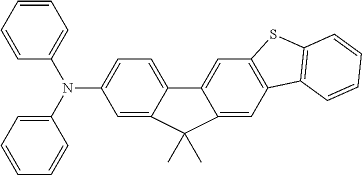

The synthesis proceeds from a 2-bromofluorene derivative. This is reacted with a 1-boronic acid 2-thioether derivative of benzene in a Suzuki coupling and oxidised. Under the influence of acid, the corresponding indeno-dibenzothiophene forms, which is oxidised using an oxidising agent. This is followed by halogenation, for example bromination, and Hartwig-Buchwald coupling in order to introduce a diarylamino group. In a final step, the sulfur is reduced again. The oxidation and reduction of the sulfur are carried out in order to selectively facilitate halogenation.

In general, the compounds according to the invention can be prepared by coupling a fluorene derivative to a benzene derivative which is substituted by a group X¹, where the group X¹ is a group which can be converted into the divalent group X, and by converting the group X¹ into the group X in a subsequent step.

The invention furthermore relates to a process for the preparation of a compound of the general formula I or II, characterised by the steps of:
a) coupling of a fluorene derivative to a benzene derivative which is substituted by a group X¹, where the group X¹ is a group which can be converted into the divalent group X, and
b) conversion of the group X¹ into the group X,

where X has the meaning indicated above.

The compounds of the formula I or II can be employed in electronic devices, in particular in organic electroluminescent devices. The precise use of the compounds depends on the substituents.

In a preferred embodiment of the invention, the compound of one of the formulae I or II is employed in an emitting layer, preferably in a mixture with at least one further compound. It is preferred for the compound of one of the formulae I or II to be the emitting compound (the dopant) in the mixture. Preferred host materials are organic compounds whose emission is of shorter wavelength than that of the compound of one of the formulae I or II or which do not emit at all.

The invention therefore furthermore relates to mixtures of one or more compounds of one of the formulae I or II with one or more host materials.

The proportion of the compound of one of the formulae I or II in the mixture of the emitting layer is between 0.1 and 99.0% by vol., preferably between 0.5 and 50.0% by vol., particularly preferably between 1.0 and 20.0% by vol., in particular between 1.0 and 10.0% by vol. Correspondingly, the proportion of the host material in the layer is between 1.0 and 99.9% by vol., preferably between 50.0 and 99.5% by vol., particularly preferably between 80.0 and 99.0% by vol., in particular between 90.0 and 99.0% by vol.

Suitable host materials are various classes of substance. Preferred host materials are selected from the classes of the oligoarylenes (for example 2, 2′,7,7′-tetraphenylspirobifluorene in accordance with EP 676461 or dinaphthylanthracene), in particular the oligoarylenes containing condensed aromatic groups, the oligoarylenevinylenes (for example DPVBi or spiro-DPVBi in accordance with EP 676461), the polypodal metal complexes (for example in accordance with WO 04/081017), the hole-conducting compounds (for example in accordance with WO 04/058911), the electron-conducting compounds, in particular ketones, phosphine oxides, sulfoxides, etc. (for example in accordance with WO 05/084081 or WO 05/084082), the atropisomers (for example in accordance with EP 1655359), the boronic acid derivatives (for example in accordance with WO 06/117052) or the benzanthracene derivatives (for example in accordance with WO 08/145,239). Particularly preferred host materials are selected from the classes of the oligoarylenes, comprising naphthalene, anthracene, benzanthracene and/or pyrene, or atropisomers of these compounds, the oligoarylenevinylenes, the ketones, the phosphine oxides and the sulfoxides. Very particularly preferred host materials are selected from the classes of the oligoarylenes, comprising naphthalene, anthracene, benzanthracene and/or pyrene, or atropisomers of these compounds.

It is furthermore particularly preferred for the compounds of one of the formulae I or II to be employed as hole-transport material and/or as hole injection material. This applies, in particular, if Y stands for N and/or if X stands for NR¹. The compounds are then preferably employed in a hole-transport layer and/or in a hole-injection layer. For the purposes of this invention, a hole-injection layer is a layer which is directly adjacent to the anode. For the purposes of this invention, a hole-transport layer is a layer which is located between the hole-injection layer and the emission layer. If the compounds of one of the formulae I or II are used as hole-transport or hole-injection material, it may be preferred for them to be doped with electron-acceptor compounds, for example with F₄-TCNQ (tetrafluorotetra-cyanoquinodimethane) or with compounds as described in EP 1476881 or EP 1596445.

If the compound of one of the formulae I or II is employed as hole-transport material in a hole-transport layer, a proportion of 100% may also be preferred, i.e. the use of this compound as pure material.

Particular preference is also given to the use of a compound of one of the formulae I or II in a hole-transport or -injection layer in combination with a layer which comprises a hexaazatriphenylene derivative, in particular hexacyanohexaazatriphenylene (for example in accordance with EP 1175470). Thus, for example, preference is given to a combination which looks as follows: anode—hexaazatriphenylene derivative—hole-transport layer, where the hole-transport layer comprises one or more compounds of the formula I or II. It is likewise possible in this structure to use a plurality of successive hole-transport layers, where at least one hole-transport layer comprises at least one compound of the formula I or II. A further preferred combination looks as follows: anode—hole-transport layer—hexaazatriphenylene derivative—hole-transport layer, where at least one of the two hole-transport layers comprises one or more compounds of the formula I or II. It is likewise possible in this structure to use a plurality of successive hole-transport layers instead of one hole-transport layer, where at least one hole-transport layer comprises at least one compound of the formula I or II.

It is furthermore preferred for the compounds of one of the formulae I or II to be employed as electron-transport material and/or as hole-blocking material for fluorescent and phosphorescent OLEDs and/or as triplet matrix material for phosphorescent OLEDs. This applies, in particular, if Y stands for C═O or P═O.

The invention furthermore relates to the use of the compounds defined above in electronic devices.

The compounds described above can also be used for the preparation of polymers, oligomers or dendrimers. This is usually carried out via polymerisable functional groups. To this end, particular preference is given to compounds which are substituted by reactive leaving groups, such as bromine, iodine, boronic acid, boronic acid ester, tosylate or triflate. These can also be used as comonomers for the generation of corresponding conjugated, partially conjugated or non-conjugated polymers, oligomers or also as the core of dendrimers. The polymerisation here is preferably carried out via the halogen functionality or the boronic acid functionality. The polymers may also have crosslinkable groups or be crosslinked via crosslinkable groups. Particularly suitable crosslinkable groups are those which are then crosslinked in the layer of the electronic device.

The invention thus furthermore relates to polymers, oligomers or dendrimers comprising one or more compounds of one of the formulae I or II. The bonds to the polymer, oligomer or dendrimer emanating from the compound of the formula I or II can be localised at any desired position of the compounds of the formula I or II which is characterised as optionally substituted by a radical R or R¹.

The polymers, oligomers or dendrimers here may be conjugated, partially conjugated or non-conjugated. Likewise encompassed are blends of the polymers, oligomers or dendrimers according to the invention with further polymers, oligomers or dendrimers.

For the purposes of this invention, the term oligomer is applied to a compound which has about three to nine recurring units. For the purposes of the invention, a polymer is taken to mean a compound which has ten or more recurring units.

The compounds according to the invention described above can be used, for example, as comonomers for the generation of corresponding conjugated, partially conjugated or non-conjugated polymers, oligomers or also as the core of dendrimers. The polymerisation here is preferably carried out via a halogen functionality and/or a boronic acid functionality.

These polymers may comprise further recurring units. These further recurring units are preferably selected from the group consisting of fluorenes (for example in accordance with EP 842208 or WO 00/22026), spirobifluorenes (for example in accordance with EP 707020, EP 894107 or EP 04028865.6), triarylamines, para-phenylenes (for example in accordance with WO 92/18552), carbazoles (for example in accordance with WO 04/070772 and WO 04/113468), thiophenes (for example in accordance with EP 1028136), dihydrophenanthrenes (for example in accordance with WO 05/014689), indenofluorenes (for example in accordance with WO 04/041901 and WO 04/113412), aromatic ketones (for example in accordance with WO 05/040302), phenanthrenes (for example in accordance with WO 05/104264) and/or metal complexes, in particular ortho-metallated iridium complexes. It should expressly be pointed out here that the polymers may also have a plurality of different recurring units selected from one or more of the groups mentioned above.

The invention likewise relates to the use of the polymers, oligomers or dendrimers defined above in electronic devices.

The invention furthermore relates to an electronic device comprising at least one compound, as defined above, or a polymer, oligomer or dendrimer, as defined above. The invention likewise encompasses blends of the oligomers, polymers or dendrimers according to the invention, optionally with further oligomers, polymers or dendrimers which are different therefrom, or further low-molecular-weight compounds.

The electronic device is preferably selected from the group consisting of organic electroluminescent devices (OLEDs), organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electro-chemical cells (LECs), organic photoreceptors and organic laser diodes (O-lasers).

For the purposes of the invention, it is preferred for the compounds of one of the formulae I or II according to the invention or the polymers, oligomers or dendrimers according to the invention to be employed as hole-transport material in a hole-transport layer and/or in a hole-injection layer in the electronic device and for it to be possible for the compounds of one of the formulae I or II or the polymers, oligomers or dendrimers in these layers to be optionally doped with electron-acceptor compounds.

For the purposes of the invention, it is furthermore preferred for the compounds of one of the formulae I or II according to the invention or the polymers, oligomers or dendrimers according to the invention to be employed as electron-transport material in an electron-transport layer and/or as hole-blocking material in a hole-blocking layer and/or as triplet matrix material in an emitting layer in the electronic device.

It is furthermore preferred for the compounds of one of the formulae I or II according to the invention or the polymers, oligomers or dendrimers according to the invention to be employed in an emission layer, preferably as emitting materials, in the electronic device.

The organic electroluminescent device comprises an anode, a cathode and at least one emitting layer, where at least one layer, which may be a hole-transport or -injection layer, an emitting layer, an electron-transport layer or another layer, comprises at least one compound of one of the formulae I or II or the polymers, oligomers or dendrimers according to the invention.

The cathode preferably comprises metals having a low work function, metal alloys or multilayered structures comprising different metals, such as, for example, alkaline-earth metals, alkali metals, main-group metals or lanthanoids (for example Ca, Ba, Mg, Al, In, Mg, Yb, Sm, etc.). In the case of multilayered structures, further metals which have a relatively high work function, such as, for example, Ag, may also be used in addition to the said metals, where combinations of the metals, such as, for example, Ca/Ag or Ba/Ag, are generally used. Preference is likewise given to metal alloys, in particular alloys comprising an alkali metal or alkaline-earth metal and silver, particularly preferably an alloy comprising Mg and Ag. It may also be preferred to introduce a thin interlayer of a material having a high dielectric constant between a metallic cathode and the organic semiconductor. Suitable for this purpose are, for example, alkali metal or alkaline-earth metal fluorides, but also the corresponding oxides or carbonates (for example LiF, Li₂O, CsF, Cs₂CO₃, BaF₂, MgO, NaF, etc.). The layer thickness of this layer is preferably between 0.5 and 5 nm.

The anode preferably comprises materials having a high work function. The anode preferably has a work function of greater than 4.5 eV vs. vacuum. Suitable for this purpose are on the one hand metals having a high redox potential, such as, for example, Ag, Pt or Au. On the other hand, metal/metal oxide electrodes (for example Al/Ni/NiOₓ, Al/PtOₓ) may also be preferred. For some applications, at least one of the electrodes must be transparent in order to enable either the irradiation of the organic material (O-SCs) or the coupling-out of light (OLEDs/PLEDs, O-lasers). A preferred structure uses a transparent anode. Preferred anode materials here are conductive mixed metal oxides. Particular preference is given to indium tin oxide (ITO) or indium zinc oxide (IZO). Preference is furthermore given to conductive, doped organic materials, in particular conductive doped polymers.

The device is correspondingly (depending on the application) structured, provided with contacts and finally hermetically sealed, since the lifetime of such devices is drastically shortened in the presence of water and/or air.

Compounds of one of the formulae I or II can also be employed either as emitting unit and/or as hole-transporting unit and/or as electron-transporting unit in polymers, oligomers or dendrimers.

Preference is furthermore given to organic electroluminescent devices, characterised in that a plurality of emitting compounds are used in the same layer or in different layers. These compounds particularly preferably have in total a plurality of emission maxima between 380 nm and 750 nm, resulting overall in white emission, i.e. at least one further emitting compound which is able to fluoresce or phosphoresce and which emits yellow, orange or red light is also used apart from the compound of one of the formulae I or II. Particular preference is given to three-layer systems, at least one layer of which comprises a compound of one of the formulae I or II and where the layers exhibit blue, green and orange or red emission (for the basic structure see, for example, WO 05/011013). Broad-band emitters can likewise be used for white-emitting OLEDs.

Apart from the cathode, anode and emitting layer, the organic electroluminescent device may also comprise further layers. These can be, for example: hole-injection layer, hole-transport layer, electron-blocking layer, hole-blocking layer, electron-transport layer, electron-injection layer and/or charge-generation layer (T. Matsumoto et al., Multiphoton Organic EL Device Having Charge Generation Layer, IDMC 2003, Taiwan; Session 21 OLED (5)). However, it should be pointed out at this point that each of these layers does not necessarily have to be present. Thus, in particular on use of compounds of one of the formulae I or II with electron-conducting host materials, very good results are furthermore obtained if the organic electroluminescent device does not comprise a separate electron-transport layer and the emitting layer is directly adjacent to the electron-injection layer or to the cathode. Alternatively, the host material may also simultaneously serve as electron-transport material in an electron-transport layer. It may likewise be preferred for the organic electroluminescent device not to comprise a separate hole-transport layer and for the emitting layer to be directly adjacent to the hole-injection layer or to the anode. It may furthermore be preferred for the compound of one of the formulae I or II simultaneously to be used as dopant in the emitting layer and as hole-conducting compound (as pure substance or as a mixture) in a hole-transport layer and/or in a hole-injection layer.

Preference is furthermore given to an organic electroluminescent device, characterised in that one or more layers are applied by means of a sublimation process, in which the materials are vapour-deposited in vacuum sublimation units at an initial pressure of less than 10⁻⁶ mbar, preferably less than 10⁻⁶ mbar. However, it should be noted that the initial pressure may also be even lower, for example less than 10⁻⁷ mbar.

Preference is likewise given to an organic electroluminescent device, characterised in that one or more layers are applied by means of the OVPD (organic vapour phase deposition) process or with the aid of carrier-gas sublimation, in which the materials are applied at a pressure of between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing or offset printing, but particularly preferably LITI (light induced thermal imaging, thermal transfer printing) or ink-jet printing. Soluble compounds of one of the formulae I or II are necessary for this purpose. High solubility can be achieved through suitable substitution of the compounds. These processes for the production of layers are particularly suitable for polymers, oligomers or dendrimers.

The compounds according to the invention preferably have one or more of the following advantages over the prior art on use in organic electroluminescent devices:
1. The power efficiency of corresponding devices becomes higher compared with systems in accordance with the prior art, in particular in the case of the use of thick layers.
2. The stability of corresponding devices becomes higher compared with systems in accordance with the prior art, which is evident, in particular, from a significantly longer lifetime, in particular in the case of the use of thick layers.
3. On use of the compounds according to the invention as hole-trans-port material in a hole-transport or hole-injection layer, it is found that the voltage is less dependent on the layer thickness of the corresponding hole-transport and/or hole-injection layer. By contrast, a greater increase in voltage is obtained with materials in accordance with the prior art in the case of relatively high layer thicknesses of the hole-transport or hole-injection layers, in turn resulting in lower power efficiency of the OLED.

4. In particular, the crystallinity of the compounds according to the invention is improved. Whereas compounds in accordance with the prior art in many cases crystallise on the vapour-deposition source during vapour deposition, which results in clogging of the source in the case of extended vapour deposition, as carried out in industrial mass production, this phenomenon is not observed at all or only to a small extent in the case of the compounds according to the invention. The compounds according to the invention are therefore particularly suitable for use in mass production.

The present application text and also the examples below are directed to the use of the compounds according to the invention in relation to OLEDs and the corresponding displays. In spite of this restriction of the description, it is possible for the person skilled in the art, without further inventive step, also to employ the compounds according to the invention for further uses in other electronic devices, for example for organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electrochemical cells (LECs), organic photoreceptors or also organic laser diodes (O-lasers), to mention but a few applications.

The present invention likewise relates to the use of the compounds according to the invention in the corresponding devices and to these devices themselves.

The invention is now explained in greater detail by the following examples, without wishing to restrict it thereby.

### EXAMPLES

The following syntheses are carried out, unless indicated otherwise, under a protective-gas atmosphere in dried solvents. The starting point used can be, for example, 9,9-dimethyl-9H-fluorene-2-boronic acid (Synlett 2006, (5), 737-740).

### Example 1

### Synthesis of Amine-1

### a) Synthesis of the (2′-nitrophenyl)fluoren-2-yl derivative

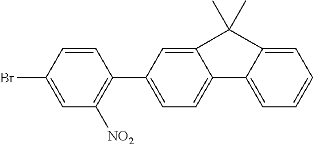

164.4 g (650 mmol) of 9,9-dimethyl-9H-fluorene-2-boronic acid, 33.8 g (124 mmol) of 2,5-dibromonitrobenzene and 164.7 g (774 mmol) of K₂CO₃ are suspended in 750 ml of THF and 750 ml of water, the mixture is saturated with N₂, 2.9 g (2.55 mmol) of tetrakis(triphenylphosphine)palladium(0) are added, and the mixture is heated at the boil for 2 h. The mixture is poured into 3 l of a mixture of water/MeOH/6 M HCl 1:1:1, and the beige precipitate is filtered off with suction, washed with water and dried. The content of product according to ¹H-NMR is about 75% with an overall yield of 183 g (90%).

### b) Synthesis of the Dibromo Derivative

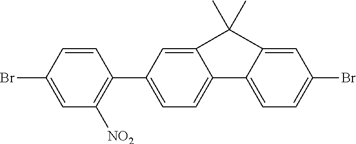

384 g (973 mmol) of the compound from a) are initially introduced in 2.5 l of chloroform under a protective gas and cooled to 5° C. 55.2 ml (1071 mmol) of Br₂, dissolved in 250 ml of chloroform, are added dropwise to this solution, and the mixture is stirred overnight. Na₂SO₃ solution is added to the mixture, the phases are separated, and the solvent is removed in vacuo. The content of product according to ¹H-NMR is about 95% with an overall yield of 400 g (86%).

### c) Synthesis of the Dibromoindenocarbazole

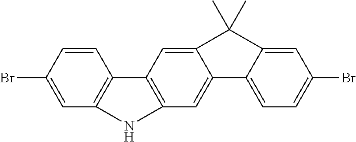

A mixture of 140 g (295 mmol) of the dibromo derivative from b) and 500 ml (2923 mmol) of triethyl phosphite is heated under reflux for 12 h. The remaining triethyl phosphite is subsequently removed by distillation (72-76° C./9 mm of Hg). Water/MeOH (1:1) is added to the residue, and the solid is filtered off and recrystallised. The content of product according to ¹H-NMR is about 96% with an overall yield of 110 g (84%).

### d) Alkylation of the Amine

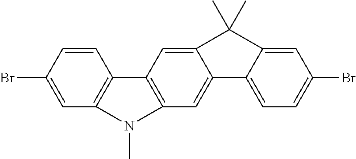

52 g (117.8 mmol) of the indenocarbazole from c) are initially introduced in 450 ml of THF and 150 ml of DMF under a protective gas and cooled to 0° C. 7 g (23 mmol) of 60% sodium hydride are added to this solution in portions. 22.01 ml of methyl iodide are subsequently added dropwise, and the mixture is allowed to come to room temperature. 200 ml of 25% NH₃ solution are added to the mixture, the phases are separated, and the solvent is removed in vacuo. The content of product according to ¹H-NMR is about 95% with an overall yield of 48.1 g (92%).

### e) Hartwig-Buchwald Coupling for the Synthesis of Amine-1

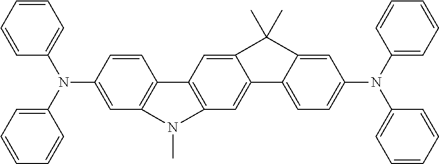

A degassed solution of 53 g (116 mmol) of the indenocarbazole from d) and 43.3 g (256 mmol) of diphenylamine in 1500 ml of dioxane is saturated with N₂ for 1 h. Firstly 11.6 ml (11.6 mmol) of 1M P(^{t}Bu)₃ solution, then 2.6 g (11.6 mmol) of palladium acetate are then added to the solution, and 33.5 g (349 mmol) of NaOtBu in the solid state are subsequently added. The reaction mixture is heated under reflux for 18 h. After cooling to room temperature, 1000 ml of water are carefully added. The organic phase is washed with 4×50 ml of H₂O, dried over MgSO₄, and the solvents are removed in vacuo. The pure product is obtained by recrystallisation. The content of product according to HPLC is 99.9% with an overall yield of 62.5 g (85%).

### Example 2

### Synthesis of Amine-2

### a) Synthesis of the Thioether

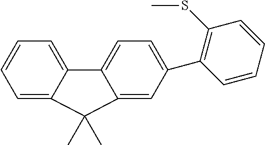

200 g (732 mmol) of 2-bromo-9,9-dimethyl-9H-fluorene, 122.9 g (732 mmol) of 2-methylsulfanylphenylboronic acid and 202 g (950 mmol) of K₂CO₃ are suspended in 850 ml of THF and 850 ml of water, the mixture is saturated with N₂, 3.3 g (2.9 mmol) of tetrakis(triphenylphosphine)palladium(0) are added, and the mixture is heated at the boil for 2 h. The mixture is poured into 31 of a mixture of water/MeOH/6 M HCl 1:1:1, and the beige precipitate is filtered off with suction, washed with water and dried. The content of product according to ¹H-NMR is about 95% with an overall yield of 190 g (82%).

### b) Oxidation of the Thioether

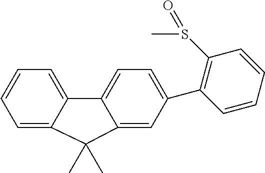

196 g (619.3 mmol) of the thioether from a) are initially introduced in 2.3 l of glacial acetic acid and 250 ml of dichloromethane under a protective gas and cooled to 0° C. 1.1 l (619 mmol) of 30% H₂O₂ solution are added dropwise to this solution, and the mixture is stirred overnight. Na₂SO₃ solution is added to the mixture, the phases are separated, and the solvent is removed in vacuo. The content of product according to ¹H-NMR is about 98% with an overall yield of 200 g (99%).

### c) Synthesis of the Indenodibenzothiophene

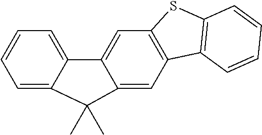

A mixture of 81 g (273 mmol) of the product from b) and 737 ml (8329 mmol) of trifluoromethanesulfonic acid is stirred at 5° C. for 48 h. 2.4 l of water/pyridine 5:1 are subsequently added to the mixture, which is then heated under reflux for 20 min. After cooling to room temperature, 500 ml of water and 1000 ml of dichloromethane are carefully added. The organic phase is washed with 4×50 ml of H₂O, dried over MgSO₄, and the solvents are removed in vacuo. The pure product is obtained by recrystallisation. The content of product according to HPLC is 98% with an overall yield of 65 g (80%).

### d) Oxidation of the Thiophene

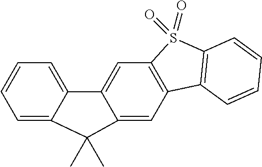

15 g (49 mmol) of the indenodibenzothiophene from c) are initially introduced in 0.3 l of glacial acetic acid under a protective gas. 33 ml (619 mmol) of 30% H₂O₂ solution are added dropwise to this solution, and the mixture is stirred overnight. Na₂SO₃ solution is added to the mixture, the phases are separated, and the solvent is removed in vacuo. The content of product according to ¹H-NMR is about 98% with an overall yield of 16 g (95%).

### e) Bromination

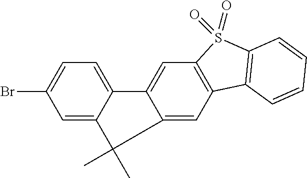

105.6 g (317.6 mmol) of the product from d) are initially introduced in 2.5 l of dichloromethane under a protective gas and cooled to 5° C. 32 ml (636.4 mmol) of Br₂ dissolved in 250 ml of chloroform are added dropwise to this solution, and the mixture is stirred overnight. Na₂SO₃ solution is added to the mixture, the phases are separated, and the solvent is removed in vacuo. The content of product according to ¹H-NMR is about 98% with an overall yield of 114 g (87%).

### f) Hartwig-Buchwald Coupling

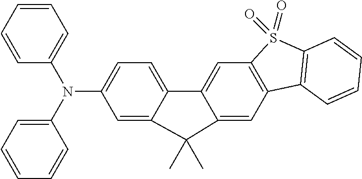

A degassed solution of 35 g (85 mmol) of the product from e) and 26 g (93 mmol) of diphenylamine in 1000 ml of dioxane is saturated with N₂ for 1 h. Firstly 0.97 ml (4.2 mmol) of P(^{t}Bu)₃, then 0.47 g (2.12 mmol) of palladium acetate are then added to the solution, and 12 g (127 mmol) of NaOtBu in the solid state are subsequently added. The reaction mixture is heated under reflux for 18 h. After cooling to room temperature, 1000 ml of water are carefully added. The organic phase is washed with 4×50 ml of H₂O, dried over MgSO₄, and the solvents are removed in vacuo. The pure product is obtained by recrystallisation. The content of product according to HPLC is 98% with an overall yield of 39 g (75%).

### g) Synthesis of Amine-2

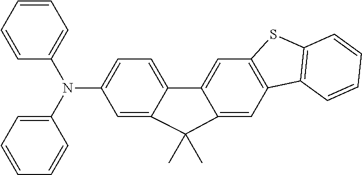

2.4 g (65 mmol) of lithium aluminium hydride are added in portions to a degassed solution of 10 g (16.3 mmol) of the product from f) in 120 ml of diethyl ether. The reaction mixture is stirred at room temperature for 2 h. 500 ml of water are then carefully added, then 250 ml of 1M HCl solution are added dropwise. The organic phase is washed with 4×50 ml of H₂O, dried over MgSO₄, and the solvents are removed in vacuo. The pure product is obtained by recrystallisation. The content of product according to HPLC is 99.9% with an overall yield of 6 g (63%).

### Examples 3 to 8

### Production of OLEDs

OLEDs according to the invention are produced by a general process in accordance with WO 04/058911, which is adapted to the circumstances described here (layer-thickness variation, materials used).

The results for various OLEDs are presented in Examples 3 to 8 below. Glass plates coated with structured ITO (indium tin oxide) form the substrates of the OLEDs. For improved processing, 20 nm of PEDOT (poly(3,4-ethylenedioxy-2,5-thiophene), applied by spin coating from water, purchased from H. C. Starck, Goslar, Germany) are applied to the substrate. The OLEDs consist of the following layer sequence: substrate/PEDOT 20 nm/HIL1 5 nm/hole-transport layer (HTM) 20, 110 or 200 nm/NPB 20 nm/emission layer (EML) 30 nm/electron-transport layer (ETM) 20 nm and finally a cathode.

The materials apart from the PEDOT are applied by thermal vapour deposition in a vacuum chamber. The emission layer here always consists of a matrix material (host) and a dopant, which is admixed with the host by coevaporation. In all the examples shown, the electron-transport layer consists of AlQ₃, and the cathode is formed by an LiF layer with a thickness of 1 nm and an aluminium layer with a thickness of 100 nm deposited on top. Table 1 shows the chemical structures of the materials used to build up the OLEDs. HTM1 here is a material in accordance with the prior art, amine-1 is an example of a compound according to the invention (synthesised in accordance with Example 1).

The OLEDs are characterised by standard methods. For this purpose, the electroluminescence spectra, the current efficiency (measured in cd/A), the power efficiency (measured in lm/W) as a function of the luminance, calculated from current-voltage-luminance characteristic lines (IUL characteristic lines), and the lifetime are determined. The lifetime is defined as the time after which the luminance has dropped to half from an initial value of 25,000 cd/m². The use voltage is defined as the voltage at which the OLED achieves a luminance of 1 cd/m².

Table 2 shows the results for some OLEDs (Examples 3 to 8). On use of the compound amine-1 according to the invention as hole-transport material in layer thicknesses of 20 and 110 nm, slightly reduced operating voltages and comparable current and power efficiencies are obtained compared with the prior art, compound HTM1 (see Examples 3, 4 and 6 and 7 from Table 2). For thicker hole-transport layers of 200 nm, which are advantageous owing to higher production yields, a significant improvement in the operating voltage is obtained on use of amine-1, giving rise to a significant increase in the power efficiency of 15% compared with the prior art (see Examples 5 and 8 in Table 2). This improvement is an extremely important aspect, especially in relation to applications in the mobile sector, since the operating duration of mobile equipment is directly dependent on the power consumption of the display. Furthermore, the compound amine-1 according to the invention is distinguished over the prior art HTM1 by the fact that, in the case of relatively thick hole-transport layers, the lifetime breaks down to a much lesser extent.

A further significant improvement with respect to processability can be achieved through the use of materials according to the invention. In this respect, FIG. 1 shows pictures of the vapour-deposition sources taken after vapour deposition of a layer of the material HTM1 in accordance with the prior art (picture a) and the material amine-1 according to the invention (picture b), each with a thickness of 700 nm. It can clearly be seen that the material HTM1 clogs the source, since a covering layer of the material forms on the upper edge of the source. As a consequence, it is only with great technical difficulty that the compound HTM1 in accordance with the prior art can be employed in mass production. By contrast, only slight edge formation on the upper part of the vapour-deposition source is obtained under the same vapour-deposition conditions (vapour-deposition rate about 1 nm/s) on use of the compound amine-1 according to the invention. This shows that materials according to the invention are significantly more suitable for mass production than are the materials in accordance with the prior art.

TABLE 1

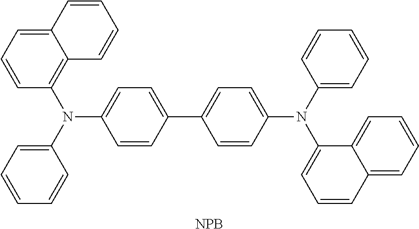

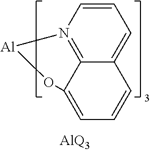

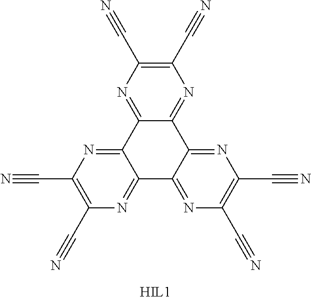

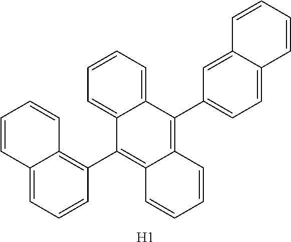

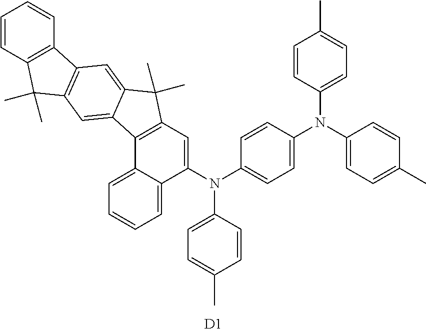

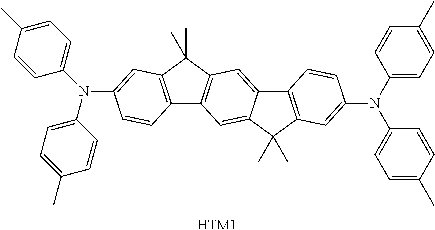

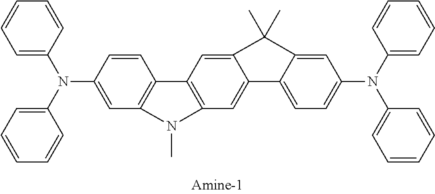

TABLE 2

| | | | | Voltage | Efficiency | Efficiency | CIE x/y | Lifetime |
|---|---|---|---|---|---|---|---|---|
| | | | Use | for 1000 | at 1000 | at 1000 | at 1000 | from 25000 |
| Ex. | EML | HTM | voltage | cd/m² | cd/m² | cd/m² | cd/m² | cd/m² |
| 3 | H1 + 10% | HTM1 | 3.2 V | 5.2 V | 14.8 cd/A | 8.9 lm/W | 0.31/0.58 | 350 h |
| (comp.) | of D1 | 20 nm | | | | | | |
| 4 | H1 + 10% | HTM1 | 3.1 V | 5.5 V | 17.1 cd/A | 9.7 lm/W | 0.29/0.61 | 243 h |
| (comp.) | of D1 | 110 nm | | | | | | |
| 5 | H1 + 10% | HTM1 | 4.1 V | 6.9 V | 15.0 cd/A | 6.8 lm/W | 0.30/0.58 | 171 h |
| (comp.) | of D1 | 200 nm | | | | | | |
| 6 | H1 + 10% | Amine-1 | 3.1 V | 5.2 V | 14.9 cd/A | 9 lm/W | 0.30/0.59 | 355 h |
| | of D1 | 20 nm | | | | | | |
| 7 | H1 + 10% | Amine-1 | 3.1 V | 5.4 V | 17.9 cd/A | 10.4 lm/W | 0.30/0.61 | 312 h |
| | of D1 | 110 nm | | | | | | |
| 8 | H1 + 10% | Amine-1 | 2.9 V | 5.9 V | 14.7 cd/A | 7.8 lm/W | 0.29/0.58 | 289 h |
| | of D1 | 200 nm | | | | | | |

### DESCRIPTION OF THE FIGURES

FIG. 1a):

Vapour-Deposition Source after Vapour Deposition of a Layer of the Material HTM1 with a Thickness of 700 nm
1=cover of the vapour-deposition source
2=upper aperture of the vapour-deposition source clogged with material

FIG. 1b):

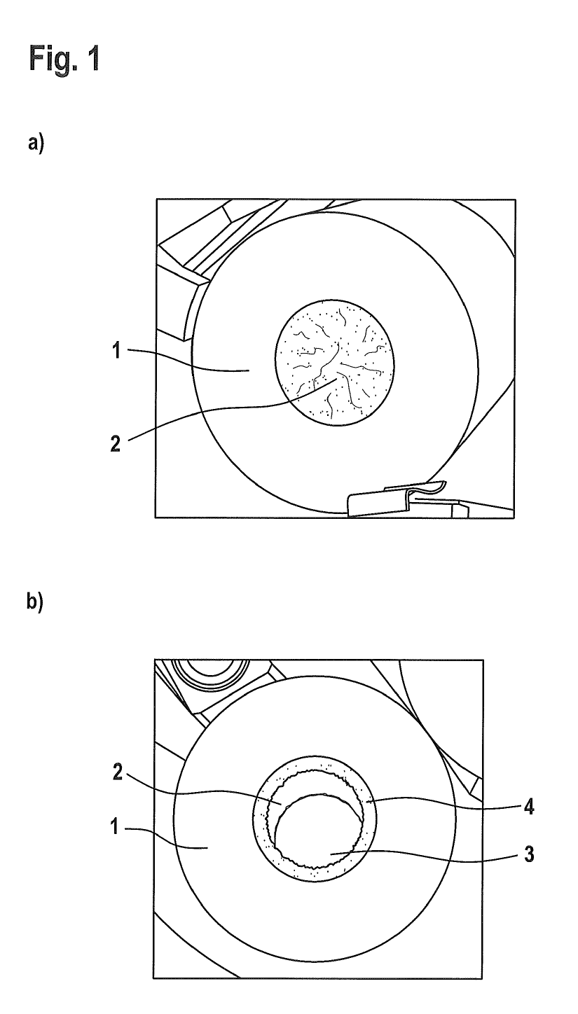

Vapour-Deposition Source after Vapour Deposition of a Layer of the Material Amine-1 with a Thickness of 700 Nm
1=cover of the vapour-deposition source
2=wall of the vapour-deposition crucible
3=residues of material on the base of the vapour-deposition crucible
4=ring of material on the upper edge of the vapour-deposition source

1-14. (canceled)

15. A compound of formula I or II 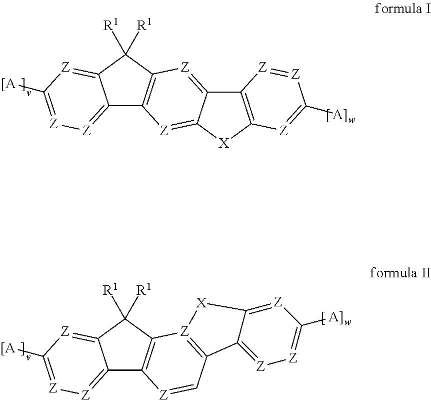
wherein
A corresponds to formula III 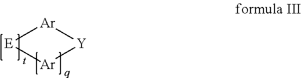
wherein the link to the compound of formula I or II takes place via Y;
Y is in each case, independently of one another, N, P, P═O, B, C═O, O, S, S═O or SO₂;
Z is in each case, independently of one another, CR or N;
X is in each case, independently of one another, a divalent bridge selected from B(R¹), C═O, C═C(R¹)₂, S, S═O, SO₂ and N(R¹);
R is in each case, independently of one another, H, D, F, Cl, Br, I, N(Ar)₂, N(R²)₂, C(═O)Ar, P(═O)Ar₂, S(═O)Ar, S(═O)₂Ar, CR²═CR²Ar, CN, NO₂, Si(R²)₃, B(OR²)₂, OSO₂R², a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which are optionally substituted by one or more radicals R², where one or more non-adjacent CH₂ groups are optionally replaced by R²C═CR², C≡C, Si(R²)₂, Ge(R²)₂, Sn(R²)₂, C═O, C═S, C═Se, C═NR², P(═O)(R²), SO, SO₂, NR², O, S or CONR² and where one or more H atoms are optionally replaced by D, F, Cl, Br, I, CN or NO₂, or an aromatic or heteroaromatic ring system having 5 to 40 aromatic ring atoms, which in each case is optionally substituted by one or more radicals R², or an aryloxy or heteroaryloxy group having 5 to 40 aromatic ring atoms, which is optionally substituted by one or more radicals R², or a combination of these systems; where, in addition, two or more substituents R optionally define a mono- or polycyclic aliphatic ring system with one another;
R¹ is in each case, independently of one another, H, D, F, Cl, Br, I, CN, NO₂, N(R²)₂, B(OR²)₂, Si(R²)₃, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which are optionally substituted by one or more radicals R², where one or more non-adjacent CH₂ groups are optionally replaced by —R²C═CR²—, —C≡C—, Si(R²)₂, Ge(R²)₂, Sn(R²)₂, C═O, C═S, C═Se, C═NR², —O—, —S—, —COO— or —CONR²— and where one or more H atoms are optionally replaced by D, F, Cl, Br, I, CN or NO₂, or arylamines or substituted carbazoles, each of which are optionally substituted by one or more radicals R², or an aromatic or heteroaromatic ring system having 5 to 40 aromatic ring atoms, which is optionally substituted by one or more non-aromatic radicals R², or an aryloxy or heteroaryloxy group having 5 to 40 aromatic ring atoms, which is optionally substituted by one or more non-aromatic radicals R², or a combination of these systems, where, in addition, two or more substituents R¹ optionally define a mono- or polycyclic ring system with one another;
R² is in each case, independently of one another, H, D or an aliphatic or aromatic hydrocarbon radical having 1 to 20 C atoms;
Ar is in each case, independently of one another, an aromatic or heteroaromatic ring system having 5 to 40 aromatic ring atoms, which is optionally substituted by one or more radicals R¹;
E is in each case, independently of one another, a single bond, N(R¹), O, S, C(R¹)₂, Si(R¹)₂ or B(R¹);
q=1 if the corresponding central atom of the group Y is an element from main group 3 or 5 or =0 if the corresponding central atom of the group Y is an element from main group 4 or 6;
t is in each case, independently of one another, 0 or 1, with the proviso that t=0 if q=0, and where t=0 means that a radical R¹ is bonded instead of the group E;
v is in each case, independently of one another, 0 or 1, with the proviso that the sum of v and w is greater than or equal to one, where v=0 means that a radical R is bonded instead of A;
w is in each case, independently of one another, 0 or 1, with the proviso that the sum of v and w is greater than or equal to one, where w=0 means that a radical R is bonded instead of A.

16. The compound of claim 15, wherein the radical Y is in each case, independently of one another, N or C═O.

17. The compound of claim 15, wherein X is selected from N(R¹) or S.

18. The compound of claim 15, wherein the group Z is in each case, independently of one another, CR.

19. The compound of claim 15, wherein Ar is phenyl, naphthyl, a substituted aromatic or heteroaromatic ring system having 5-15 carbon atoms or an aromatic or heteroaromatic ring system which is substituted by arylamine or carbazole.

20. The compound of claim 15, wherein E is not present, i.e. t=0, or where E is a single bond or C(R¹)₂.

21. The compound of claim 15, wherein the compound is selected from the formulae Ia and IIa:

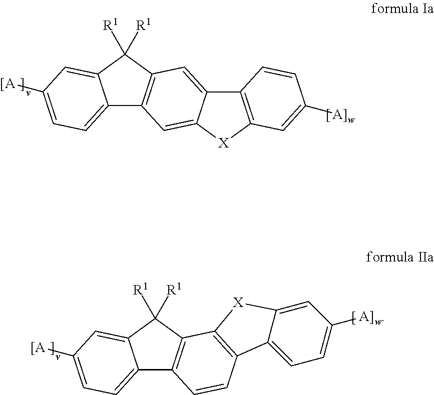

22. The compound of claim 21, wherein X is equal to S or N(R¹).

23. A polymer, oligomer, or dendrimer comprising one or more compounds of claim 15, wherein the bonds to the polymer, oligomer or dendrimer emanating from the compound of formula I or II can be localised at any desired position of the compound of formula I or II, wherein said one or more compounds are optionally substituted by a radical R or R¹.

24. An electronic device comprising at least one compound of claim 15.

25. The electronic device of claim 24, wherein said device is selected from the group consisting of organic electroluminescent devices, organic field-effect transistors, organic thin-film transistors, organic light-emitting transistors, organic integrated circuits, organic solar cells, organic field-quench devices, light-emitting electrochemical cells, organic photoreceptors, and organic laser diodes.

26. The electronic device of claim 24, wherein said device is an organic electroluminescent device wherein said at least one compound of claim 15 is employed therein as a hole-transport material in a hole-transport layer and/or hole-injection layer, wherein the compounds in these layers are optionally doped with electron-acceptor compounds, or wherein said at least one compound of claim 15 is employed as electron-transport material in an electron-transport layer and/or as hole-blocking material in a hole-blocking layer and/or as triplet matrix material in an emitting layer, or wherein said at least one compound of claim 15 is employed in an emitting layer.

27. A process for preparing the compound of claim 15, comprising the steps of:
a) coupling of a fluorene derivative to a benzene derivative which is substituted by a group X¹, where the group X¹ is a group which can be converted into the divalent group X, and
b) conversion of the group X¹ into the group X.
